(19) United States
(12) Patent Application Publication
(10) Pub. No.: US 2018/0118747 A1
(43) Pub. Date: May 3, 2018

(54) METHOD OF ISOLATING THEACRINE AND COMPOSITION COMPRISING THEACRINE

(71) Applicant: ThermoLife International, LLC, Phoenix, AZ (US)

(72) Inventors: Ronald Kramer, Phoenix, AZ (US); Alexandros Nikolaidis, Nea Kallikratia (GR)

(73) Assignee: ThermoLife International, LLC, Phoenix, AZ (US)

(21) Appl. No.: 15/861,284

(22) Filed: Jan. 3, 2018

Related U.S. Application Data
(60) Provisional application No. 62/441,886, filed on Jan. 3, 2017.

(51) Int. Cl. C07D 473/14; B01F 3/20; B01F 3/22

(52) U.S. Cl. CPC C07D 473/14; B01F 3/2078; B01F 2003/005; B01F 3/2261; B01F 3/2276; B01F 3/2215

(57) ABSTRACT

Disclosed are improved methods for producing solid powdered theacrine compositions that are virtually flavorless and improved theacrine compositions.

### CROSS REFERENCE TO RELATED APPLICATIONS

This document claims the benefit of the filing date of U.S. Provisional Patent Application No. 62/441,866 titled “METHOD OF ISOLATING THEACRINE AND COMPOSITION COMPRISING THEACRINE” to Alexander Nikolaidis and Ronald Kramer, which was filed on Jan. 3, 2017, the disclosure of which is hereby incorporated entirely herein by reference.

### BACKGROUND

Theacrine, also known as 1,3,7,9-tetramethyluric acid, is a purine alkaloid found in Cupuaçu (Theobroma grandiflorum) and in Camellia assamica var. kucha (a tea known as “ku ding cha”). It has anti-inflammatory and analgesic effects and appears to affect adenosine signaling in a manner similar to caffeine. In kucha leaves, theacrine is synthesized from caffeine in what is thought to be a three-step pathway. Recently, theacrine has gained a lot of interest in the nutritional supplements industry, as recent studies indicate that it may enhance energy and reduce fatigue.

Despite the growing positive data about theacrine, its use in sports supplements is still limited. A major reason is the extremely bitter taste of theacrine. This bitter taste is difficult to mask, and it makes all solutions containing theacrine unpalatable. In fact, the translation of the Chinese name for the type of tea made with kucha leaves is “bitter green tea.” Because of the prominent bitterness in taste, the amount of theacrine that has been used in powdered/liquid products is small and limited, for example to 25 mg per serving of powdered supplement. Even then, the powdered/liquid products require heavy use of flavoring to mask the bitter taste from theacrine. Thus, in order to increase the use and quantity of theacrine into more types of supplements, the bitterness of theacrine needs to be reduced without impacting its anti-inflammatory and caffeine-like effects.

### SUMMARY

This disclosure relates to methods of reducing bitterness of theacrine, wherein the methods produce solid theacrine compositions.

In one aspect, the method includes: heating a carrier to its melting point temperature to produce a melted carrier; dissolving powdered theacrine; mixing the dissolved powdered theacrine into the melted carrier to produce a mixture; cooling the mixture; milling the cooled mixture to a desirable particle size; and washing off, under filtration, the milled mixture with a solvent until the washed off solute has no detectable bitter flavor. The solvent solubilizes theacrine not fused with the carrier. The method may further include gathering the washed off solute and evaporating the gathered washed off solute to recover dissolved theacrine.

In another aspect, the method includes: dissolving a carrier in a first solvent selected from the group consisting of: an alcohol and water; dissolving powdered theacrine in the first solvent; mixing the dissolved carrier and the dissolved powdered theacrine to produce a mixture; evaporating the first solvent from the mixture; milling the evaporated mixture in a desirable particle size; and washing off under filtration the milled mixture with second solvent selected from the group consisting of: ethanol, chloroform, methanol, and water until the washed off solute has no detectable bitter flavor, wherein the second solvent solubilizes theacrine. The first solvent may be evaporated under vacuum condition.

In some implementations, the first solvent may be ethanol, and in some implementations, the second solvent may be water.

The carrier may be shellac and the solvent may be water made alkaline. The water is made alkaline with a suitable base that is suitable for human consumption, such as, sodium bicarbonate, calcium hydroxide, magnesium hydroxide and the like.

The method may further include gathering the washed off solute and evaporating the gathered washed off solute to recover dissolved theacrine.

This disclosure also relates to a solid composition comprising theacrine or its derivatives and a carrier, for example, the solid compositions produced according to the methods described above and in the Detailed Description.

In one aspect, compositions may include theacrine dispersed inside the composition, for example, evenly dispersed among the carrier. The carrier is selected from the group consisting of: a high molecular weight fatty acid, hydrogenated derivative of the high molecular weight fatty acid, esterified derivative of the high molecular weight fatty acid, an aliphatic alcohol, hydrogenated derivative of the aliphatic alcohol, esterified derivative of the aliphatic alcohol, resin, wax, and mixtures thereof. In some aspects, the carrier comprises the majority of the composition by weight.

The amount of theacrine in the composition may be 0.01%-50% by weight of the composition. Specifically, theacrine may be 20% by weight of the composition.

In some embodiments, the carrier may be a high molecular weight fatty acid, such as stearic acid. In other embodiments, the carrier may be a composition consisting of 80% by weight stearic acid and 20% by weight carnauba wax. In other embodiments, the carrier may be palmitic acid. In still other embodiments, the carrier may be shellac.

### DETAILED DESCRIPTION

The present disclosure is directed to methods for producing solid (at the usual storage temperature: 0° C.-50° C.) powdered theacrine compositions that are virtually flavorless. Thus, the methods also are directed to reducing or eliminating the bitterness of theacrine. Such theacrine compositions are suitable for use in oral supplements where the bitter taste of theacrine would not be desirable. Such oral supplements may be in the form of liquids (for example ready-to-drink drinks), powders, chewable tablets, candies, and the like.

As used herein, the term “theacrine” refers to free form theacrine as well as all its derivative forms, which includes but are not limited to: salts (e.g. theacrine nitrate and theacrine hydrochloride) and N-substituted derivatives (e.g. 1,3,7,-trimethyluric acid and 1,3,7,9-tetraethyluric acid).

As used herein, the term “water” refer to unaltered water, which would have a pH of around 7 and up to 7.4. As used herein, “water made alkaline” or “water at alkaline pH” or “alkalized water” refers to water with pH that is higher than 7.4. “Water made alkaline” or “water at alkaline pH” or “alkalized water” could be the result of mixing water with a suitable base, for example, sodium bicarbonate, calcium hydroxide, or magnesium hydroxide.

As used herein, the term “majority” refers to a greater quantity or share. In some aspects, majority refers to greater than half of the total.

Unlike methods that require microencapsulation coating of bitter ingredients, which is an expensive and tricky process, the disclosed method is inexpensive, easy to perform, and produces superior results in the reduction of bitterness. Theacrine treated with this method also lasts longer and overall produces better results than free form theacrine. For example, theacrine treated with this method results in longer effects on the central nervous system.

The methods of invention involve fusing the theacrine with a carrier. Generally, the methods comprise mixing theacrine and a carrier in a liquid environment. The liquid environment may be provided by melting the carrier. Alternatively, the liquid environment may be a solvent that dissolves both the carrier and theacrine, for example, ethanol, water-ethanol mixtures, alkalized water acetone and ether. After the carrier and theacrine are mixed, the mixture is cooled and/or dried to a solid then milled to the desired particle size. After which, the milled solid is washed with a solvent in which theacrine is soluble but not the carrier, for example water. The washing step is performed with a filter to separate the milled solid from the washed off solute. This may also be performed under vacuum conditions. The washing step is performed until the washed off solute has no detectable bitter flavor. At this point, all of the uncoated theacrine was been washed away to leave only coated theacrine in the milled solid. In some implementations, the washed off solute is collected and dried to collect the uncoated theacrine.

The carrier must be solid at room temperature, preferably up to 45° C. The carrier also does not have a melting temperature not exceeding 295° C. Importantly, the carrier must be suitable for human consumption. Preferably, the carrier should also be soluble in evaporable solvents, have no offensive taste, be insoluble in water or saliva, and have no chemical incompatibilities with theacrine.

As such, the examples of a suitable carrier include high molecular weight fatty acids, for example, Lauric (C12), Tridecylic (C13), Myristic (C14), Pentadecanoic (C15), Palmitic (C16), Margaric (C17), Stearic (C18), Nonadecylic (C19), Arachidic (C20), Heneicosylic (C21), Behenic (C22), Tricosylic (C23), Lignoceric (C24), Pentacosylic (C25), Cerotic (C26), Heptacosylic (C27), Montanic (C28), Nonacosylic (C29), Melissic (C30), Hentriacontylic (C31), Lacceroic (C32), Psyllic (C33), Geddic (C34), Ceroplastic (C35), Hexatriacontylic (C36), Heptatriacontanoic (C37), Octatriacontanoic (C38). The carrier may also be a hydrogenated monounsaturated and polyunsaturated high molecular weight fatty acid, such as hydrogenated soybean oil. High molecular weight alcohols, such as stearyl alcohol and lauryl alcohol, may be a carrier. Esters of aforementioned alcohols and acids, such as lauryl stearate, may also be a carrier. Waxes, such as bee wax and carnauba wax, and resins, such as shellac and mastic gum, can also be used as a carrier. The carrier may also be a composition of the aforementioned examples. Therefore, the carrier may be selected from the group consisting of: a high molecular weight fatty acid, hydrogenated derivative of the high molecular weight fatty acid, esterified derivative of the high molecular weight fatty acid, an aliphatic alcohol, hydrogenated derivative of the aliphatic alcohol, esterified derivative of the aliphatic alcohol, resin, wax, and mixtures thereof

Accordingly, the composition of the invention comprises theacrine or its derivatives; and a carrier, wherein theacrine is dispersed inside the composition. Such a composition has reduced bitter flavor compared to theacrine. In some aspects, the theacrine is evenly dispersed in the composition, for example among the carrier. As explained in the methods above, theacrine may be evenly dispersed among the carrier by mixing powdered theacrine with melted carrier or dissolved carrier. The composition may also include other additives that can enhance the pharmaceutical or organoleptic properties, (e.g. one of a solubilizer, an enzyme inhibiting agent, an anticoagulant, an antifoaming agent, an antioxidant, a coloring agent, a coolant, a cryoprotectant, a hydrogen bonding agent, a flavoring agent, a plasticizer, a preservative, a sweetener, a thickener, and combinations thereof) and/or a second carrier (e.g. one of an excipient, a lubricant, a binder, a disintegrator, a diluent, an extender, a solvent, a suspending agent, a dissolution aid, an isotonization agent, a buffering agent, a soothing agent, an amphipathic lipid delivery system, and combinations thereof). These additives may be solids or liquids, and the type of additive may be generally chosen based on the type of administration being used. Those of ordinary skill in the art will be able to readily select suitable pharmaceutically effective additives from the disclosure in this document. In particular implementations, pharmaceutically acceptable additives may include, by non-limiting example, calcium phosphate, cellulose, stearic acid, croscarmelose cellulose, magnesium stearate, and silicon dioxide.

The person well versed in the art will understand that other ingredients that are typically included in theacrine nutraceutical formulations may be fused together in the composition. Such ingredients include but are not limited to caffeine and other natural stimulants such as synephrine, amino acids and their derivatives, inorganic nitrates, vitamins, herbal extracts, and the like.

### EXAMPLES

The present disclosure is further illustrated by the following examples that should not be construed as limiting. The contents of all references, patents, and published patent applications cited throughout this application, as well as the Figures, are incorporated herein by reference in their entirety for all purposes.

I. A Sample Preparation Method Using Stearic Acid.

Stearic acid is safe to use in foods, relatively inexpensive, stable in storage, practically insoluble in water. It has a melting point of 69.3° C., which is not so hot as to destroy many temperature-sensitive ingredients. Below is a sample method where the carrier is stearic acid.

1. Measure 80 grams of stearic acid and 20 grams of theacrine free base.

2. Melt stearic acid at 69.3° C.

3. Slowly pour the theacrine into the melted stearic acid. Mix continuously for at least 5 minutes.

4. Cool down the mixture of theacrine and stearic acid.

5. Mill the mixture into fine powder using a pharmaceutical mill.

6. Put the powder in a filter funnel and wash off under vacuum with water for 5 minutes.

7. Air dry the powder.

II. A Sample Method of Using Shellac to Procure the Tasteless Bitter Theacrine Composition:

1. Dissolve 50 grams shellac and 20 grams of theacrine in 250 ml of ethanol.

2. Mix until both the shellac and the theacrine are totally dissolved.

3. Evaporate the ethanol at 60° C. under vacuum until a solid is formed.

4. Mill the solid into powder.

5. Wash the powder on a paper filter with water for at least 5 minutes.

III. Improved Effects of Fused Theacrine:

Typically, theacrine's effects in human subjects start after 30 minutes of ingestion and last 6-8 hours. However, when the level of theacrine in the human subject is reduced to where the nerves are not saturated with theacrine, the human subject feels a “crash.” However, when the same amount of theacrine was ingested in the form of fused theacrine of the disclosure, the human subjects reports that the effects of theacrine was felt for 8-12 hours and did not feel a “crash.” The fused theacrine results is prolonged gradual release of theacrine.

1. A method of reducing or eliminating the bitterness of theacrine, said method comprising:
heating a carrier until it reaches its melting temperature point to produce a melted carrier;
dissolving powdered theacrine;
mixing the dissolved powdered theacrine into the melted carrier to produce a mixture;
cooling the mixture;
milling the cooled mixture to a desirable particle size; and
washing off under filtration the milled mixture with a solvent until the washed off solute has no detectable bitter flavor, wherein the solvent solubilizes theacrine.

2. The method of claim 1, further comprising gathering the washed off solute and evaporating the gathered washed off solute to recover dissolved theacrine.

3. A method of reducing or eliminating the bitterness of theacrine, said method consists of:
dissolving a carrier in a first solvent selected from the group consisting of: an alcohol and water;
dissolving powdered theacrine in the first solvent;
mixing the dissolved carrier and the dissolved powdered theacrine to produce a mixture;
evaporating the first solvent from the mixture;
milling the evaporated mixture in a desirable particle size; and
washing off under filtration the milled mixture with second solvent selected from the group consisting of: ethanol, chloroform, methanol, and water until the washed off solute has no detectable bitter flavor, wherein the second solvent solubilizes theacrine.

4. The method of claim 3, wherein the first solvent is evaporated under vacuum condition.

5. The method of claim 3, wherein the first solvent is ethanol.

6. The method of claim 3, wherein the second solvent is water.

7. The method of claim 3, further consisting gathering the washed off solute and evaporating the gathered washed off solute to recover dissolved theacrine.

8. The method of claim 3, wherein the carrier is shellac and the solvent is water made alkaline with sodium bicarbonate.

9. A solid composition produced according to the method of claim 1.

10. A solid composition produced according to the method of claim 3.

11. A solid composition comprising:
theacrine or its derivatives; and
a carrier selected from the group consisting of: a high molecular weight fatty acid, hydrogenated derivative of the high molecular weight fatty acid, esterified derivative of the high molecular weight fatty acid, an aliphatic alcohol, hydrogenated derivative of the aliphatic alcohol, esterified derivative of the aliphatic alcohol, resin, wax, and mixtures thereof,
wherein theacrine is dispersed inside the composition and said composition has reduced bitter flavor.

12. The composition of claim 11, wherein the fatty acid is stearic acid.

13. The composition of claim 12, wherein theacrine in an amount of 0.01%-50% by weight of the composition.

14. The composition of claim 12, wherein theacrine is 20% by weight of the composition.

15. The composition of claim 11, wherein the carrier comprises the majority of the composition by weight.

16. The composition of claim 15, wherein the carrier is selected from the group consisting of: stearic acid, palmitic acid, resin, and shellac.

17. The composition of claim 11, wherein the carrier is a composition consisting of 80% by weight stearic acid and 20% by weight carnauba wax.

18. The composition of claim 11, wherein the carrier is selected from the group consisting of palmitic acid, resin, and shellac.

19. The composition of claim 11, wherein theacrine is evenly dispersed among the carrier by mixing theacrine powder in the melted carrier.

20. The composition of claim 11, wherein theacrine is evenly dispersed among the carrier by mixing theacrine powder in the dissolved carrier.
